(19) United States
(12) Patent Application Publication
(10) Pub. No.: US 2017/0189578 A1
(43) Pub. Date: Jul. 6, 2017

(54) BONE MATRIX WITH CHANNELS TO DELIVER DESIRED AMOUNT OF HYDRATION, METHOD OF MAKING AND METHOD OF USING THE SAME

(71) Applicants: Daniel Cox, Bozeman, MT (US); Michael Mansfield, Bozeman, MT (US); Helena Lovick, Great Falls, MT (US)

(72) Inventors: Daniel Cox, Bozeman, MT (US); Michael Mansfield, Bozeman, MT (US); Helena Lovick, Great Falls, MT (US)

(21) Appl. No.: 15/397,619

(22) Filed: Jan. 3, 2017

Related U.S. Application Data
(60) Provisional application No. 62/273,777, filed on Dec. 31, 2015.

(51) Int. Cl. A61L 27/36; A61L 27/56; A61L 27/54

(52) U.S. Cl. CPC A61L 27/3608; A61L 27/54; A61L 27/56; A61L 2430/02

(57) ABSTRACT

The present invention relates to shaped, bone-based products for the delivery of a measured quantity of biologic material for use. The products can be used alone or in combination with a biologic material as a surgical implant. The present invention also relates to a method to make the product and a method of using the product.

### CROSS REFERENCE TO RELATED APPLICATIONS

This application claims priority and the benefit under 35 U.S.C. §119(e) to U.S. Provisional Patent Application Ser. No. 62/273,777 filed Dec. 31, 2015, which is incorporated herein in its entirety by reference.

### FIELD OF THE INVENTION

The present embodiments generally relate to bone-based products sized and/or shaped to receive a selected quantity of a biologic material. The products are meant to be used alone or in combination with an added biologic material as a composite surgical implant.

### BACKGROUND

Methods for manufacturing shaped bone-based products are known in the prior art. These methods include the use of subtractive milling of natural bone segments, cutting and sizing of bone fragments, and the addition of various carriers and excipients to generate products of the desired shape and physical characteristics. It is known that the addition of excess biologic material to a bone scaffold may be detrimental. For example, both the debris from graft disintegration and the debris from cellular die-off can lead to post-operative inflammation and serve as a potential infection site. Accordingly, such debris may impede the overall bone healing process (for more information, see Claes L, et al. “Fracture healing under healthy and inflammatory conditions” Nat Rev Rheumatol. 2012;8(3):133-143). In another example, the presence of excess growth factors (e.g., rhBMP-2) during spinal fusion procedures has produced numerous reports of increased rates of ectopic bone formation (for example, see Simmonds M C, et al. “Safety and Effectiveness of Recombinant Human Bone Morphogenetic Protein-2 for Spinal Fusion” Ann Intern Med. 2013;158(12): 877-889, and Hustedt J W, et al. “The controversy surrounding bone morphogenetic proteins in the spine: a review of current research” Yale J Biol Med. 2014;87(4):549-561).

U.S. Pat. No. 6,541,024 to Kadiyala et al., which is incorporated in its entirety by reference, discloses compositions and methods for augmenting bone formation by administering mesenchymal stem cells in combination with a resorbable biopolymer. U.S. Pat. No. 6,395,311 to Qi, which is incorporated in its entirety by reference, discloses a vehicle for the delivery of biological active agents. The vehicle is specifically formulated from a combination of plant extracts comprising aloe vera polysaccharide. U.S. Patent Publication No. 2007/0160622 to Turnell, et al., which is incorporated in its entirety by reference, details methods for assembling a polymer-biologic composition for delivery of a therapeutic biologic. A preferred composition includes a synthetic biodegradable polymer pre-attached to a metal affinity ligand, non-covalently complexed with a transition metal ion.

U.S. Pat. No. 8,980,248 to Shoichet et al., which is incorporated in its entirety by reference, claims an injectable cell delivery composition comprising a polymer composition and at least one cell type for delivery. The hydrogel system disclosed has applications for minimally invasive cell delivery to the body.

A need remains for a bone-based implant of the correct shape or format to receive a proper quantity of a biologic material.

### SUMMARY OF THE INVENTION

The disclosed invention is directed to bone-based implants for bone fusion and bone regeneration where the implant is selectively sized and/or shaped to receive a designated volume of a biologic material. By restricting the ratio of the bone-based implant material to the volume of a biologic material, the composite implants display enhanced tissue regenerative effects. Furthermore, by tuning into a suitable volume to bone implant ratio, the resultant composite implants have more consistent and predictable physical characteristics for the surgeon end user. The present invention discloses bone-based implant products that are advantageous over the prior art as discussed below.

The use of channels and/or voids can assist in hydrating the product at a faster rate compared to a product not containing channels and/or voids for a given volume of bone material.

An aspect of the invention is a shaped, bone-based product. The product includes at least one channel and/or void. The dimensions of the channel are selected to form a proper volume and porosity to receive at least one biologic material.

An aspect of the invention is a method to form a shaped, bone-based product. The product includes at least one channel and/or void of a proper volume and porosity to receive at least one biologic material.

An aspect of the invention is a method of using a shaped bone based implant product. The product includes at least one void or channel for providing a rehydrating fluid to the product.

### BRIEF DESCRIPTION OF THE FIGURES

FIG. 1 illustrates a perspective view of a bone-based implant in accordance with the present invention; and

FIG. 2 illustrates a perspective, time-lapse view of a bone-based implant absorbing a quantity of a blood substitute.

### DETAILED DESCRIPTION OF THE INVENTION

The present invention relates to bone-based products for the delivery of a quantity of biologic material for surgical implantation. The products can be used alone or in combination with a biologic material as a surgical implant.

“Allogeneic” or “allograft”, as used herein, refers to tissue derived from a non-identical donor of the same species, which can be a DBM.

“Autogeneic” or “autograft”, as used herein, refers to tissue derived from and implanted into the same identical patient.

“Biocompatible”, as used herein, refers to the property of being biologically compatible with a living being by not causing harm.

“Osteoinductive”, as used herein, refers to the ability of a material to induce bone healing via recruitment of osteoprogenitor cells.

“Patient”, as used herein, refers to a living recipient of the biomaterial-based implants of the present invention.

“Xenogeneic” or “xenograft”, as used herein, is defined as tissue derived from a non-identical donor of a different species.

An aspect of the invention is a shaped, bone-based product. The product includes at least one channel. The dimensions of the channel are selected to form a proper volume and porosity to receive at least one biologic material.

More porous channels increase the product's surface area to expose more material to the added biologic material. Macro porosity (pore sizes of about 100-3000 μm) increases cellular ingrowth. In some embodiments, the porosity of the product ranges from about 20% to about 99%. In some embodiments, the porosity of the product can be about 20%, about 30%, about 40%, about 50%, about 60%, about 70%, about 80%, about 90% or about 99% In some embodiments the pore size of the product range from about 5 to about 3000 μm. In some embodiments, the pore size of the product can be between about 100 and 2000 μm, between about 250 and about 1750 μm, or between about 500 and 1000 μm.

The bone-based products can include a single material or a mixture of materials which can be used as a scaffold during bone regrowth. In some embodiments, the shaped product can be comprised solely of bone tissue. The bone tissue can be cortical bone, cancellous bone, or a combination thereof. The bone tissue can be fully mineralized, partially demineralized, or fully demineralized. In some embodiments, the bone tissue can be demineralized so that the residual calcium is less than 8% by weight. The bone can be allogeneic, autogeneic, xenogeneic tissues, or combinations thereof. In other embodiments, the bone material can be combined with other materials. Other suitable materials to be combined with the bone material of the product include, but are not limited to, biocompatible and/or biodegradable materials. Biocompatible materials can include, but are not limited to plastics, polymers, metals, ceramics, and combinations thereof Biodegradable materials can include but are not limited to, collagens, gelatins, biodegradable plastics, biodegradable polymers, biological tissues, and combinations thereof.

The bone-based implants can contain bone fibers, bone particles, bone sponges, or a combination of these bone forms. “Bone fibers”, as used herein, refers to bone pieces in the shape of a thread or narrow strip of bone. The average length of the bone fibers can be about 1 mm to about 200 mm and the average width of the bone fiber can be about 10 μm to about 2 mm. In some embodiments, the average length of the bone fibers can be between about 50 mm and about 150 mm, or between about 75 mm and about 100 mm. The average width of the bone fibers can be between about 50 μm and about 1 mm, or between about 75 μm and about 1.5 mm. “Bone particles”, as used herein, refers to bone pieces in the shape of granules, regular or irregular in shape. Example shapes of bone particles include spheres, spheroids, cubes, and so forth. Bone particles can range in diameter from about 10 μm to about 2 mm. In some embodiments, the diameter of the bone particle can between about 50 μm and about 1 mm, or between about 75 μm and about 1.5 mm. “Bone sponges”, as used herein, refers to bone pieces originating from trabecular or “spongy” bone. Bone sponges are softer and more flexible than bone pieces originating from cortical bone. The bone-based implants can be provided to the end user and can be used as a biologic or cellular delivery vehicle to enhance bone regeneration, bone fusion, and healing. In some embodiments, the bone-based implants can be dehydrated prior to use. The bone-based implants can be shaped to fit or contained within a medical device apparatus or implantation site. In some embodiments, the medical device apparatus can be a spinal fusion cage.

The implant can be combined with biologic materials. Suitable biologic materials include, but are not limited to, bone marrow aspirate, whole blood, plasma, serum albumin, concentrated bone marrow aspirate, platelet rich plasma, mesenchymal stem cells, osteogenic cell lines, chondrogenic cell lines, connective tissue progenitor cells, and combinations thereof.

In some embodiments, the bone materials can be exposed to drying or lyophilization conditions. In some embodiments, the bone materials can be shaped and sized to specific dimensions to enhance entanglement and subsequent final product self-adhesion, flexibility, and compressibility. The bone-based implants can be any suitable shape including, but not limited to, a cube, a block, a strip, or a sphere.

The void (created during manufacturing) to bone material ratio of the product can be between about 1:99 to about 1:11, to provide a porous bone material. In some embodiments, the void to bone ratio can be about 1:99, about 1:90, about 1:80, about 1:70, about 1:60, about 1:50,about 1:40, about 1:30, about 1:20, or about 1:11. In some embodiments, the void to bone material can range from about 50-95% porosity.

The channel/void to porous bone material ratio of the product, (wherein the porous bone material includes the volume of the bone material and the voids contained therein) can be between about 15:1 to 1:1, in some embodiments between about 1:10 to about 10:1. In some embodiments, the ratio of channels/voids to bone material is between about 4:1 and about 1:4. In some embodiments, the channel/void to porous bone material ratio of the product can be about 10:1, about 9:1, about 8:1, about 7:1, about 6:1, 5:1, about 4:1, about 3:1, about 2:1, about 1:1, about 1:2, about 1:3, about 1:4, about 1:5, about 1:6, about 1:7, about 1:8, about 1:9, about 1:10, about 2:5, or about 3:7. In some embodiments, the channel/void can constitute at least about 25% of the bone material. In some embodiments, the channel/voids can constitute about 25% of the bone product, wherein the bone product is between about 50-95% porous.

In some embodiments, the bone materials can be combined with bioactive agents, crystalloids, colloids, polymers, carriers, combinations thereof, or other agents to facilitate cohesion, flexibility and compressibility of the bone-based implants. Bioactive agents include bioactive glass composites, calcium phosphates, biphasic calcium phosphates, hydroxyapatities and combinations thereof. Crystalloids include dextrose, lactose, hydroxyethyl starch, trehalose, sucrose, salines, phosphates, and combinations thereof. Colloids include gelatin, blood, albumin, gelofusine, xanthan gum, and combinations thereof. Polymers include polylactides, polyglycolides, polyethylene glycols, poloxamers, carboxymethyl cellulose, combinations thereof, and copolymers thereof. Carriers include, glycerol, mineral oil and combinations thereof. In some embodiments, the bone material can be combined with bioactive agents, crystalloids, colloids, polymers, carriers, or other agents, in singular form or in combination. The ratio of additives to bone material can range from about 2:1 to about 1:10. In some embodiments, the ratio of additives to bone material can be about 2:1, about 3:1, about 4:1, about 5:1, about 6:1, about 7:1, about 8:1, about 9:1 or about 10:1.

The residual moisture content of the product can be less than about 20%. In some embodiments, the residual moisture content of the product can be less than about 20%, less than about 15%, less than about 10%, or less than about 6%.

The rehydrated product can be compressed to about 20% of an original size of the product before dehydration. In some embodiments, the rehydrated product can be compressed to about 20% of an original size, about 15% of an original size, or about 10% of an original size. In some embodiments following compression, the rehydrated product can return to its original shape of the product before dehydration. The rehydrated product can be moldable into a shape other than the dehydrated product shape.

The product can be partially dehydrated, fully dehydrated, or fully hydrated. When dehydrated, the bone-based implant product can be rehydrated in at least one aqueous liquid and can rehydrate within about 15 minutes. The aqueous liquid can contain water, saline, buffer, balanced salt solution, bone marrow aspirate, plasma, serum albumin, or combinations thereof. In some embodiments, the aqueous liquid can contain growth factors, hormones, bioactive agents, cells, or combinations thereof. Growth factors and hormones can include, but are not limited to, calcium regulating hormones, parathyroid hormone (PHT), transforming growth factor beta (TGF-beta), prostaglandin E2 (PGE2), bone morphogenetic proteins (BMPs, e.g., BMP-2), insulin like growth factors (IGFs, e.g., IGF-1), and combinations thereof. Bioactive agents can include, but are not limited to, antibiotics, bioactive glasses, bioactive minerals, and combinations thereof. The cells can include mesenchymal stem cells, blood cells, osteogenic cell lines, chondrogenic cell lines, and combinations thereof. The biologic material within the aqueous liquid can be at a specified concentration.

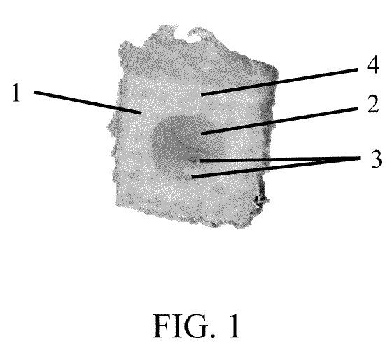

FIG. 1 illustrates a bone-based product 1 in accordance with the present invention. The products can be formed into shapes including, but not limited to, cylinders, cubes, blocks, strips, spheres, discs, and doughnuts. In some embodiments, the product can be shaped specifically to fill a bone void. The void can be determined by pre-assessment of a void, such as a bone void within a patient. The final use of the product can be placement within a void of a patient. The products can be provided hydrated, partially dehydrated, or fully dehydrated. In some embodiments, the products can be dehydrated to contain less than about 6% residual moisture by weight. The products can be dried by lyophilization, dehydration, convection drying, or conventional heating methods, or combinations of these drying methods.

The products can be provided with at least one void 2 at some location within the product 1. The product 1 can contain at least one channel 3 or other such depression, such as a purposeful void 2. A channel as used herein means purposeful opening for bulk flow of the biologic material throughout the channel. A void as used herein means open space within the product wherein the biologic material can pass through via capillary action or other diffusion process. The void in the product for receiving a biologic material should not be confused with the voids existing from the manufacturing. The void 2 and channel 3 serve to increase the product surface area for biologic material adsorption and absorption. The void 2 and channel 3 within the product 1 can be shaped in a manner to receive an exact volume or quantity of a biologic material. In some embodiments, the specific quantity of biologic material can be added in excess and the void 2 and/or channel 3 in the product 1 can release the excess, unneeded quantity of biologic material. FIG. 1 illustrates channel 3 which function as drainage holes or openings to allow a biologic material to enter and exit the product during use. As used herein, reference to a void or channel, such as void 2 or channel 3 refers to contiguous open spaces within a product, as distinct from space between bone fibers or bone particles or within a bone sponge, such as interstitial spaces. In some embodiments, the product 1 can be designed to receive a specific quantity of a biologic material determined by the mass of the product 1.

The amount of biologic material can be provided in a volume ratio of biologic material to porous bone-based product. The volumetric ratio of the biologic material to the porous bone-based product can range from about 10:1 to about 1:1, about 8:1 to about 2:1, or about 4:1, wherein the volume of the porous bone-based product includes only the volume of the bone material and the contained void and pore spaces, but excludes the volume of the channel spaces. The use of the proper volume ratio of biologic material to bone-based product can serve to enhance the tissue regenerative ability of the bone-based product. Voids 2 and/or channels 3 within a product 1 can also be characterized by their void to bone-based product ratios. For example, voids and/or channels can have void to product ratios ranging from about 1:15 to about 4:1, about 1:10 to about 2:1, or about 1:8 to about 1:1. The total amount of biologic material provided to the product 1 can be provided by one or more voids 2 and/or channels 3. Voids 2 and/or channels 3 within a product 1 can also be characterized by the percent of implant porosity (e.g., via mercury porosimetry testing). The voids 2 and channels 3 within the product 1 can serve to retain, attract, and attach biologic materials to the product 1 surface. The product 1 can further have surface depressions 4 and/or irregularities to enhance the product's osteoconductive surface. Channels 3 on the product 1 can fully or partially traverse the product's outer surfaces. The channels 3 of the product 1 can be shaped to draw biologic material containing liquids through the product 1 by capillary action.

An aspect of the invention is a method to form a shaped, bone-based product. The product includes at least one channel of a proper volume and porosity to receive at least one biologic material.

The bone-based material of the products can be manufactured in a specific shape and composition as described in U.S. Publication No. 2015/0251361 entitled “Shaped Fiber-Based Products and a Method of Manufacture Thereof” (“the 361 Publication”), which is incorporated by reference in its entirety. The bone-based material of the products can be cut to a desired size and manufactured as described in U.S. Pat. No. 8,574,825 “Process for Demineralization of Bone Matrix with Preservation of Natural Growth Factors,” which is incorporated by reference in its entirety.

The voids or channels can be formed during the manufacturing process of the implants. By way of example only, the voids or channels can be formed with the use of a mold used to prevent material from occupying the space. In some embodiments, the voids or channels can be formed following the formation of the product by removing material from the product. In some embodiments, the size of the voids or channels can be formed using a mold and further adjusted by removing additional material from the product or by adding material to the product. Methods for removing material from the product can include, but are not limited to, drilling, milling, cutting, and boring. Methods for adding material to the product can include, but are not limited to, three-dimensional printing, melt-bonding, press-fitting, vapor deposition, and liquid deposition.

The voids or channels can be of any geometry without limitation. For example, the shape can be selected from a capsule, a circular cone, a circular cylinder, a hemisphere, a pyramid, a rectangular prism, a spherical cap, a spherical segment, a tube, a disc, a donut, a block, a strip, a cube, a sphere, or any portion of the foregoing.

An aspect of the invention is a method of using a shaped bone based implant product. The product includes at least one void or channel for providing a rehydrating fluid to the product.

The dehydrated or partially dehydrated bone-based products can rehydrate rapidly within a hydration fluid over a period of about 15 seconds to about 30 minutes, of about 1 minute to about 25 minutes, or of about 5 minutes to about 20 minutes. In some embodiments, the dehydrated or partially dehydrated bone-based products can also have a high rehydration rate of between about 0.5 mL of liquid/g of products/minute to about 10 mL of liquid/g of product/minute. Other methods to dehydrate the product and particular conditions, including temperature, pressure, etc. are discussed in the '361 Publication, which has been incorporated by reference in its entirety. Hydration fluids can further contain a biologic material. Suitable hydration fluids include, but are not limited to, water, salines, phosphate buffered salines, buffers, balanced salt solutions, blood, bone marrow aspirate, plasma, serum albumin and combinations thereof.

The products can be combined with a biologic material prior to, or during use. Suitable biologic materials include, but are not limited to, bone marrow aspirate, whole blood, plasma, serum albumin, concentrated bone marrow aspirate, platelet rich plasma, mesenchymal stem cells, osteogenic cell lines, chondrogenic cell lines, connective tissue progenitor cells, and combinations thereof.

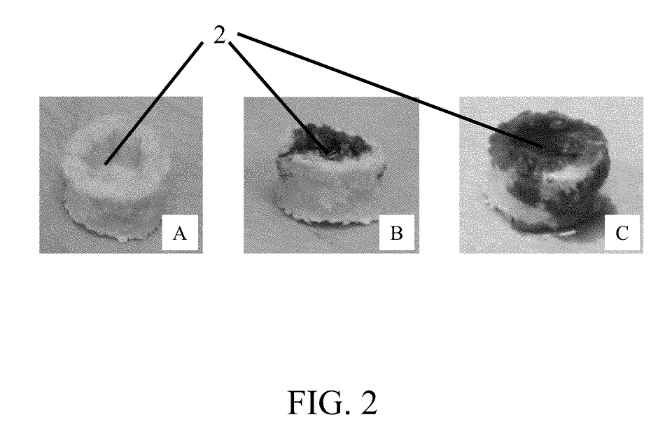

FIG. 2 illustrates a product manufactured in accordance with the present invention. The product starts in dehydrated form (FIG. 2A), a hydrating agent, illustrated in FIG. 2B as a blood substitute is added to the void 2 in the product in a 4:1 product:volume ratio. The product is shown to absorb and disseminate the blood substitute throughout the article in FIG. 2C.

In some embodiments, the implants can be further handled post-hydration to a desired consistency. The desired consistency of the implants can be putty-like with a viscosity of about 200 to 100,000,000 centipoise, of about 5,000 to 50,000,000 centipoise.

In some embodiments, the implant can be placed within an external implant or cage. The external implant or cage can be a medical device. The external implant or cage can be composed of biocompatible materials.

Ranges have been discussed and used within the forgoing description. One skilled in the art would understand that any sub-range within the stated range would be suitable, as would any number within the broad range, without deviating from the invention.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A shaped product comprising a bone material wherein the product is configured to define at least one channel and/or void having a volume to receive at least one biologic material.

2. The product of claim 1, wherein the shaped product is selected from the group consisting of a cylinder, a cube, a block, a strip, a sphere, a disc, a doughnut and combinations thereof.

3. The product of claim 1, wherein a shape of the at least one channel and/or void is selected from the group consisting of a cylinder, a cone, a cube, a block, a strip, a sphere, a disc, a doughnut and combinations thereof

4. The product of claim 1, wherein the at least one biologic material is selected from the group consisting of a bone marrow aspirate, a serum albumin, a whole blood, a plasma, a concentrated bone marrow aspirate, a platelet rich plasma, and combinations thereof

5. The product of claim 1, wherein the volume of the at least one channel and/or void to receive the at least one biologic material and a volume of the shaped, bone-based product have an established ratio.

6. The product of claim 5, wherein the established ratio is between about 10:1 and about 1:1.

7. The product of claim 1, wherein the product receives the biologic material, and wherein the biologic material is selected from the group consisting of a bone marrow aspirate, a whole blood, a serum albumin, a plasma, a concentrated bone marrow aspirate, a platelet rich plasma, and combinations thereof.

8. The product of claim 1, wherein the bone material is mineralized, partially demineralized, fully demineralized, or combinations thereof.

9. The product of claim 1, wherein the bone material is selected from the group consisting of bone fibers, bone particles, a bone sponge, and combinations thereof.

10. The product of claim 1, wherein the product further comprises at least one bioactive agent, at least one crystalloid, at least one colloid, at least one polymer, and at least one carrier.

11. The product of claim 1, wherein the product is at least partially dehydrated and a residual moisture content of the product is less than about 20%.

12. The product of claim 1, wherein the product is compressible to about 10% of a pre-dehydration size.

13. The product of claim 1, wherein a ratio of void (porosity) to the bone material of the product is between about 1:99 and about 1:11 to provide a porous bone material.

14. The product of claim 1, wherein a material of the bone is an allogeneic tissue, an autogeneic tissue, a xenogeneic tissue, or combinations thereof.

15. The product of claim 13, wherein the at least one channel and/or void to porous bone material ratio is between about 15:1 and about 1:1.

16. A method to form a shaped product, comprising: forming a shaped product, wherein the shaped product comprises at least one channel and/or void, and wherein the at least one channel and/or void provides a rehydrating fluid to the product.

17. The method of claim 16, wherein the at least one channel and/or void are formed with a mold.

18. The method of claim 16, wherein the at least one channel and/or void are formed after the product is formed by removing material of the product.

19. A method to implant a shaped bone based implant product, wherein the product comprises at least one channel and/or void, comprising:
providing a rehydrating fluid to the at least one void or channel;
rehydrating the product; and
providing the bone product to a patient.

20. The method claim 19, further comprising providing the product is provided to the patient using an implant tool.
